(19) United States
(12) Patent Application Publication
(10) Pub. No.: US 2018/0368910 A1
(43) Pub. Date: Dec. 27, 2018

(54) Electrosurgical Forceps with Embedded Heat Pipe

(71) Applicant: Kirwan Surgical Products LLC, Marshfield, MA (US)

(72) Inventors: Lawrence T. KIRWAN, JR., Kingston, MA (US); Chadwick J. WHITCHER, Kingston, MA (US); Jerry R. GRIFFITHS, Norwell, MA (US)

(21) Appl. No.: 16/016,816

(22) Filed: Jun. 25, 2018

Related U.S. Application Data
(60) Provisional application No. 62/524,673, filed on Jun. 26, 2017.

(51) Int. Cl. A61B 18/14

(52) U.S. Cl. CPC A61B 18/1445; A61B 2018/00041

(57) ABSTRACT

An electrosurgical forceps having an enhanced capability to transfer heat from the tip to eliminate or minimize sticking of tissue is provided. A heat pipe is secured within a groove of at least one tine of the forceps. The heat pipe has an evaporator section in thermal communication with the tip, and a condenser section in thermal communication with a portion of the body, which serves as a heat sink to receive heat transferred from the tip. A method of manufacturing the electrosurgical forceps is also provided.

### CROSS REFERENCE TO RELATED APPLICATIONS

This application claims priority under 35 U.S.C. § 119(e) of U.S. Provisional Application No. 62/524,673, filed on Jun. 26, 2017, entitled “Electrosurgical Forceps with Embedded Heat Pipe,” the disclosure of which is hereby incorporated by reference.

### STATEMENT REGARDING FEDERALLY SPONSORED RESEARCH OR DEVELOPMENT

N/A

### BACKGROUND

Electrosurgical forceps have a pair of resilient blades or arms that are used for grasping and coagulating tissue. The forceps may be monopolar or bipolar. In monopolar forceps, the blades are welded or otherwise joined to form an electrode in electrical communication with an electrosurgical generator. Current flows from the active electrode through the patient's tissue to a dispersive electrode in contact with the patient's skin (which may be at some distance from the forceps) and back to the generator. In bipolar forceps, each blade of the pair comprises an electrode in communication with an electrosurgical generator. Current flows from one blade through the tissue to the other blade.

In some instances, tissue may stick to the tips of the blades. If sticking occurs, the surgeon must pull on the forceps to release it from the tissue, possibly causing further bleeding and requiring that the forceps be cleaned. It is known to prevent or minimize such sticking of tissue to electrosurgical forceps by manufacturing the blades of the forceps from a material that minimizes sticking, such as nickel. See, for example, U.S. Pat. Nos. 5,196,009 and 6,059,783.

Another known manner of preventing or minimizing sticking is to form the blades from a metal or metal alloy having a relatively high thermal conductivity, such as copper, that is able to transfer heat away from the tips of the blades. By keeping the tissue cooler, coagulation is able to occur without sticking of the tissue. Nickel is more biocompatible with human tissue than copper and is preferable for contact with tissue, as well as providing additional non-stick capabilities. Thus, a known forceps provides blades formed of an inner layer of copper or copper alloy having a thickness sufficient to dissipate heat and an outer covering of a strong, biocompatible metal or metal alloy such as nickel. See U.S. Pat. Nos. 6,059,783 and 6,298,550.

Other electrosurgical forceps that prevent or minimize sticking to tissue are known. U.S. Pat. No. 6,749,610 discloses an electrosurgical forceps in which at least one of the tines has an outer plating that covers all or substantially all of the tine. The outer plating includes silver, rhodium, gold, aluminum, palladium, tungsten, or nickel. U.S. Pat. Nos. 7,789,882, 8,108,994, and 8,656,585 disclose electrosurgical forceps in which a tip is formed of a composite material having aligned elongated nickel particles interspersed in a matrix of silver particles and a tip or blade member formed of a dispersion strengthened silver or copper composite material.

### SUMMARY OF THE INVENTION

The present invention relates to electrosurgical forceps having an enhanced capability to transfer heat away from the tip of the forceps by embedding a heat pipe in one or both tines. The non-stick properties of the forceps are thereby enhanced. A method of manufacturing electrosurgical forceps with an embedded heat pipe is also provided.

The heat pipe can be secured within a groove in a surface of one or both tines of the forceps, thereby allowing the heat pipe to employ all or substantially all of the adjacent body of the tine to transfer heat away from the tip. The amount of surface area of the heat pipe in thermal communication with the body of the tine can be maximized. The heat pipe can be permanently affixed within the tine. The forceps with the embedded heat pipe can be reusable or disposable.

### DESCRIPTION OF THE DRAWINGS

The invention will be more fully understood from the following detailed description taken in conjunction with the accompanying drawings in which:

FIG. 1 is an isometric view of an embodiment of electrosurgical forceps with an embedded heat pipe;

FIG. 2 is a side view of the forceps of FIG. 1;

FIG. 3 is a plan view of a blank with embedded heat pipe for one tine of the forceps of FIG. 1;

FIG. 4 is a plan view of a blank with embedded heat pipe for another tine of the forceps of FIG. 1;

FIG. 5 is a cross-sectional view along line V-V of FIG. 6 of an embodiment of a heat pipe for use with the forceps of FIG. 1;

FIG. 6 is a schematic illustration of an embodiment of a heat pipe for use with the forceps of FIG. 1;

FIG. 7 is a cross-sectional view of a further embodiment of a heat pipe for use with the forceps of FIG. 1;

FIG. 8 is a plan view of a blank with embedded forceps for one tine of a further embodiment of forceps; and

FIG. 9 is a plan view of a blank with embedded forceps for another tine of the embodiment of FIG. 8.

### DETAILED DESCRIPTION OF THE INVENTION

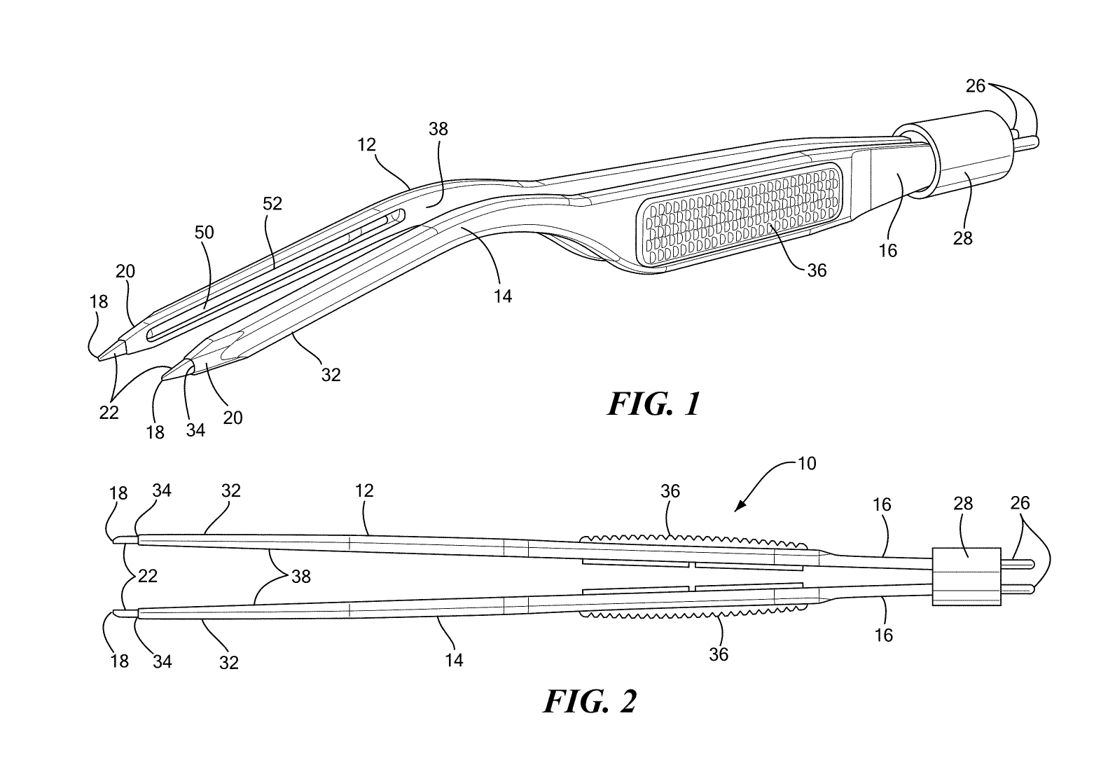

Referring to FIGS. 1 and 2, an electrosurgical forceps 10 has first and second tines or blade members 12, 14 constructed from an electrically and thermally conducting material. In some embodiments, the material can be one or more metallic materials, such as stainless steel, or aluminum, copper, nickel, titanium, or alloys thereof. In some embodiments, the material can be one or more composite metal materials, such as nickel particles interspersed in a silver matrix or dispersion strengthened silver or copper. In some embodiments, the tines can be constructed in a layered configuration. Each of the tines 12, 14 is elongated and extends from a first or proximal end 16 to a second or distal end 18 having a tip 20. The tines are generally flat to have a greater width than depth, such that the tips are configured for gripping tissue between opposed surfaces 22. The length of the tip for grasping tissue can be about ⅜ inch, and can range from about 0.25 inch to 0.5 inch, although other lengths can be provided. A plating of an electrically and thermally conductive biocompatible material such as gold may be provided on the tip or extending along a portion of the length or the entire length of each tine. In the embodiment illustrated, the tip and the body of the tine are integrally formed from a unitary piece of material. In some embodiments, the tip can be attached to the body of the tine in any suitable manner, such as by brazing. In some embodiments, the tip and/or the body of the tine can be formed of a composite material having aligned elongated nickel particles interspersed in a matrix of silver particles. In some embodiments, the tip and/or the body of the tine can be formed of a dispersion strengthened silver or copper composite material.

Proximal ends 16 are electrically connected in any suitable manner, such as by crimping, welding, or soldering, to terminal pins 26. The proximal ends 16 along with the terminal pins 26 are encapsulated using an epoxy-based material or otherwise mounted within an insulating cap portion 28. In some embodiments, the tines 12, 14 can be insulated with an insulating material 32 along most of their length from the cap portion 28 to a location 34 adjacent to the tip 20. In some embodiments, gripping pads 36 can be attached to each tine 12, 14 at a suitable location to aid a user in gripping the forceps during use. In some embodiments, finger grips, for example, serrations (not shown), may be formed on each tine at a suitable gripping location. Bipolar forceps are shown; however, the forceps could be monopolar forceps as well.

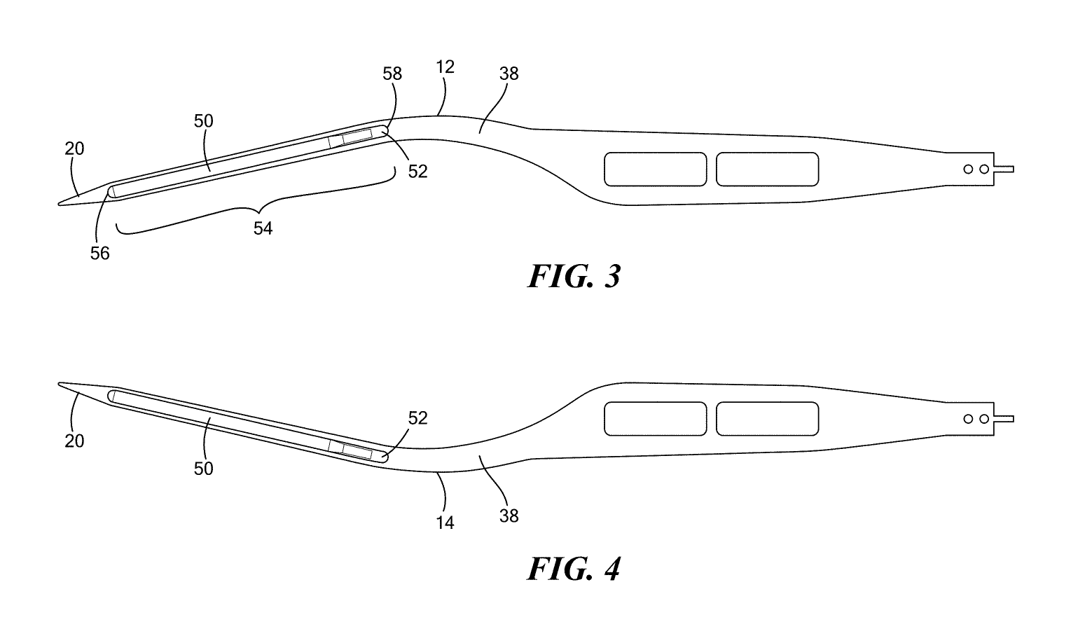

A heat pipe 50 is disposed in a groove 52 in an inner facing surface 38 that extends along a portion 54 of the tine from a location 56 in thermal communication with the tip 20 to a location 58 spaced from the tip, such as in a midsection of the tine. See FIGS. 3 and 4. The heat pipe, described with more particularity below, is partially filled with a working fluid that vaporizes adjacent to the tip and condenses at a location spaced from the tip, thereby transferring heat away from the tip and to the body of the tine. Because the full working length of the heat pipe can be in thermal communication with the body of the tine, the thermally conductive material of the tine can act as a heat sink to disperse heat throughout the body of the tine. In some embodiments, the heat pipe can have a round or circular cross-section. In some embodiments, the heat pipe can have a flattened, oval, elliptical, or other cross-section, which can further increase the surface area available for thermal communication. The heat pipe can be secured within the groove, as by press fitting and/or with a thermal interface material 62, such as a thermal epoxy. The groove can be configured to conform to the cross-sectional configuration of the heat pipe. The thermal interface material can also minimize air gaps within the groove around the heat pipe. The thermal interface material can also overlie the entire heat pipe. The thermal interface material thereby helps to provide good thermal contact between the heat pipe and the body of the tine. In some embodiments, the heat pipe can fit entirely within the groove, at or below the surface of the tine. The insulation material can also cover the heat pipe and the thermal interface material. In this manner, no portion of the heat pipe extends above the inner surface of the tine, thereby maximizing the surface area of the heat pipe that is in thermal communication with the tine. Also, the heat pipe does not interfere with the line of sight of the user.

This configuration allows the heat pipe to employ all or substantially all of the adjacent body of the tine to transfer heat away from the tip. The amount of surface area of the heat pipe in thermal communication with the body of the tine can be maximized. The heat pipe is not limited to transferring heat from the tip to a heat sink displaced at a distance away from the tip. In some embodiments, the heat pipe can be permanently affixed within the tine. In some embodiments, the forceps with the embedded heat pipe can be reusable. In some embodiments, the forceps with the embedded heat pipe can be disposable. In some embodiments, heat pipe can be disposed in a groove in an outer facing surface. In some embodiments, the forceps can be monopolar and a single heat pipe can be provided in one of the tines.

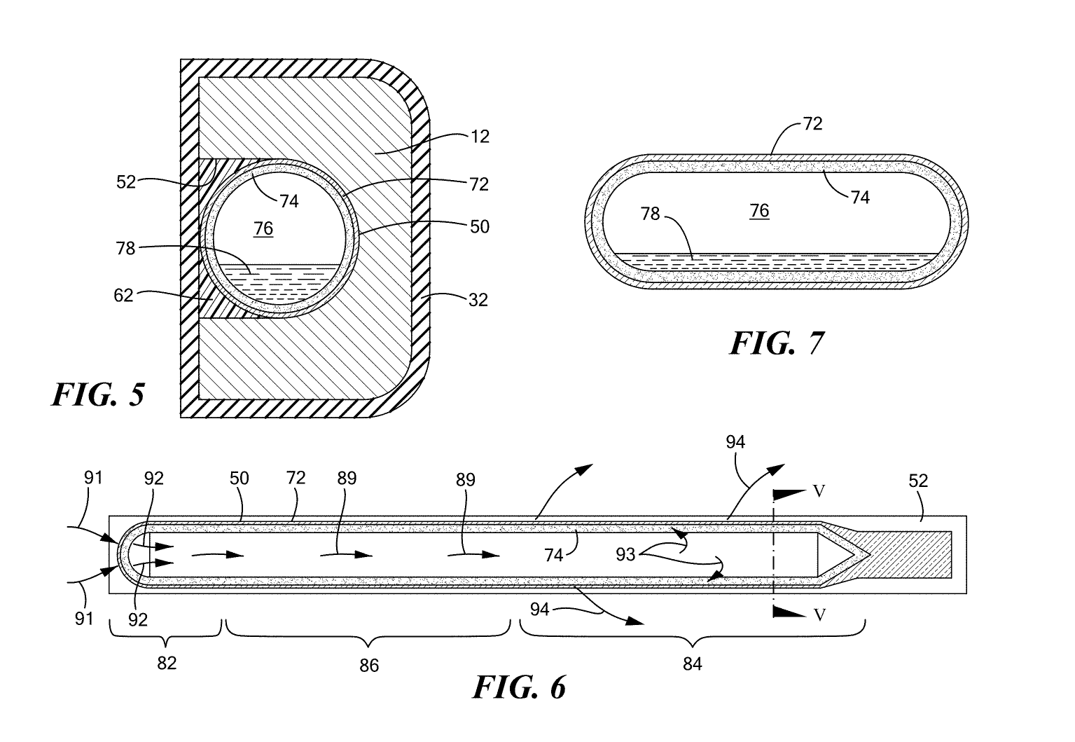

Referring more particularly to FIGS. 5 and 6, the heat pipe 50 can be a constant conductance heat pipe that includes an elongated sealed casing 72 extending from a higher temperature evaporator section 82 to a lower temperature condenser section 84. An adiabatic section 86 is disposed between the evaporator section and the condenser section. It will be appreciated that the boundaries between these sections are indicated in FIG. 6 only approximately for purposes of explanation. In use, the evaporator section is placed in thermal contact with a heat source, i.e., the tip of the tine, and the condenser section is placed in thermal contact with a heat sink, i.e., a portion of the body of the tine spaced proximally from the tip. A wicking structure 74 is disposed within the casing, for example, lining an interior surface of the casing along its length. The wicking structure provides a plurality of capillary pathways through which a working fluid 78 can travel as a liquid from the condenser section to the evaporator section. The casing includes an open region 76 within the wicking structure, for example, a central core coaxial with a wicking structure lining the casing. The open region provides a pathway for the working fluid 78 to flow as a gas from the evaporator section to the condenser section (indicated schematically by arrows 89).

The working fluid 78 is enclosed within the sealed casing 72. At the evaporator section, heat transfer from the heat source, the forceps tip, to the working fluid (indicated schematically by arrows 91) causes a phase change from liquid to gas, such that the working fluid in liquid phase in the wicking structure evaporates and enters the core as a gas (indicated schematically by arrows 92). At the condenser section, fluid in the gas phase in the core condenses to the liquid phase and enters the wicking structure (indicated schematically by arrows 93). Heat is transferred, via the phase change to a liquid, out of the heat pipe at the condenser section to the body of the tine (indicated schematically by arrows 94). A vapor pressure differential between the hotter evaporator section and the cooler condenser section causes the working fluid to travel as a gas from the evaporator section through the open region to the condenser section. Liquid enters into the wicking structure at the condenser section to return by capillary force to the evaporator section. The pressure difference between the liquid and vapor phases is maintained by the surface tension of the liquid phase in the wicking structure. In use, the forceps can typically be oriented such that gravity can assist in movement of the working fluid. The evaporated fluid generally travels up, and the condensed fluid travels down.

In some embodiments, the length of the heat pipe can range from about 2 inches to about 4 inches. In some embodiments, the heat pipe can have a circular cross-section, as shown in FIG. 5, and its diameter can range from about 0.06 inch to about 0.10 inch. In some embodiments, the heat pipe can have an oval or elliptical cross-section; the width can range from about 0.08 inch to about 0.13 inch, and the height can range from about 0.04 inch to about 0.08 inch. See FIG. 7. In some embodiments, the front or distal end of the heat pipe is in thermal contact with the proximal end of the tip over a surface area of about 0.1 in². The condenser section can be relatively long to maximize heat transfer to the body of the tine, since the entire heat pipe length is in contact with the body. No additional section of the heat pipe is needed for attachment to the tine.

In some embodiments, the heat pipe can operate between a temperature range of about 80° C. to about 100° C. at the high temperature end to a temperature at ambient or slightly above ambient at the low temperature end. In some embodiments, the working fluid can be distilled water.

In some embodiments, the thermally conductive material of the heat pipe casing 72 can be copper or a copper alloy. In some embodiments, the casing material can be aluminum or nickel or alloys thereof. In some embodiments, the wicking structure can be formed of a sintered powder metal. In some embodiments, the wicking material can be formed of the same metal material as the casing. In some embodiments, a suitable thermal interface material can be a thermal epoxy or thermal solder. Some commercially available heat pipes, such as heat pipes used for computer cooling, can be used.

The forceps can be manufactured in any suitable manner. In some embodiments, a sheet of appropriate material for the forceps tines is cut into strips. Each strip is cut to an appropriate length for a tine. A taper is stamped at one end of the strip for the tip of the tine. A groove is milled in the strip for the heat pipe. Apertures are cut in a mid portion of the strip for receiving the gripping pads, or serrations for a finger grip are stamped into the mid portion. In a next step, the rear or spring section is cold formed, as by rolling, to compress its thickness and to work harden the material. Work hardening of the material in this section strengthens the material, enabling a user to squeeze the tines together repeatedly to grasp tissue and release the tines to return to their rest position. The perimeter of the strip is stamped to form the general shape of the blade member. The tine could have bends along its length, as shown in FIGS. 1 and 2, or could have a generally straight configuration, depending on the particular application. In a next step, the perimeter of the tine is formed, as with a coining process, to form the edges.

A tab is stamped, deburred, and formed at the end of the tine. The terminal pins may be attached to the tabs in any suitable manner, such as by crimping, welding, or soldering. Holes may be stamped into the end to allow epoxy or other appropriate potting material to flow through and around the tines to fix the tines more firmly within the cap portion. The tip is plated with a thin layer of an electrically and thermally conducting, biocompatible material, such as gold, using conventional plating processes.

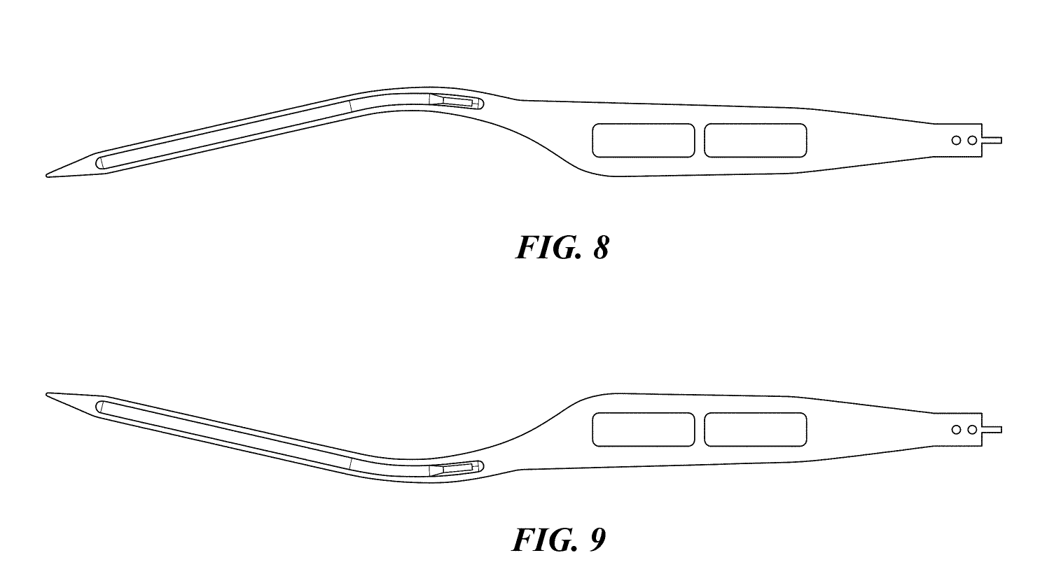

A thermal interface material 62, such as a thermal epoxy, is applied in the groove, and the heat pipe is press fit into the groove. In some embodiments, the heat pipe can be formed, as by bending, to fit the shape of the forceps tine, as shown in FIGS. 8 and 9. Additional thermal interface material can be applied over the heat pipe flush with the surface of the tine. In this manner, air gaps within the groove around the heat pipe can be minimized and the surface area of the heat pipe in thermal communication with the body of the forceps tine can be maximized. The tine is then insulated along most of its length, which also covers the heat pipe. The insulation material can include, without limitation, a polypropylene (PP), a polyvinylidene fluoride (PVDF), a polyamide, or a polyethylene (PE). The insulation may be formed in any suitable manner, such as by spraying on a liquid that dries to form a solid coating. The tip of the tine is left uninsulated for a suitable distance, such as about ⅜ inch. The insulation is typically 0.010 to 0.015 inches thick.

Further aspects and embodiments of the invention include the following:

1. An electrosurgical forceps comprising:

an insulated cap portion;

at least one electrical terminal extending from and fixed to the cap portion;

a pair of tines, each tine comprising a generally elongated body, a tip located at a distal end of the tine, and a proximal end of the tine fixed within the cap portion, at least one tine of the pair of tines electrically connected to the at least one electrical terminal within the cap portion, the body of the at least one tine comprising an electrically conductive and thermally conductive material, and having a groove formed within a surface of the body extending from the tip to a location at a midportion of the body; and

a heat pipe secured within the groove of the at least one tine, the heat pipe comprising a sealed casing and a working fluid within the casing for fluid flow between a condenser section and an evaporator section, the evaporator section disposed in thermal communication with the tip, and the condenser section disposed in thermal communication with a portion of the body of the at least one tine at a location proximally spaced from the tip.

2. The forceps of embodiment 1, further comprising a thermal interface material within the groove in thermal communication between at least portions of the heat pipe and the body of the at least one tine.
3. The forceps of any of embodiments 1-2, wherein the groove is disposed in an inwardly facing surface of the at least one tine.
4. The forceps of any of embodiments 1-3, wherein the heat pipe is straight.
5. The forceps of any of embodiments 1-4, wherein the heat pipe includes a curved or bent section conforming to a curve or bend in the at least one tine.
6. The forceps of any of embodiments 1-5, wherein the heat pipe has a circular, oval, elliptical, or flattened cross section.
7. The forceps of any of embodiments 1-6, wherein the casing of the heat pipe is copper or a copper alloy.
8. The forceps of any of embodiments 1-7, wherein the working fluid is water.
9. The forceps of any of embodiments 1-8, wherein the heat pipe includes a wicking structure within the casing, the wicking structure comprising a plurality of capillary channels for liquid flow of the working fluid from the condenser section to the evaporator section.
10. The forceps of any of embodiments 1-9, further comprising an insulating coating over the body of the tine extending from the cap portion to a location adjacent to the tip, the insulating coating extending over the heat pipe.
11. The forceps of any of embodiments 1-10, further comprising a second heat pipe secured within a groove in a second tine of the pair of tines.
12. The forceps of any of embodiments 1-11, wherein the electrically conductive and thermally conductive material of the body of the at least one tine is a stainless steel or aluminum, copper, nickel, titanium, or an alloy thereof.
13. The forceps of any of embodiments 1-12, wherein the tip or the body of the at least one tine or both the tip and the body comprise a composite material, wherein the composite material is a dispersion strengthened silver or copper composite material.
14. The forceps of any of embodiments 1-13, wherein the tip or the body of the at least one tine or both the tip and the body comprise a composite material having aligned elongated nickel particles interspersed in a matrix of silver particles.
15. The forceps of any of embodiments 1-14, wherein the forceps is a bipolar forceps.
16. A method of manufacturing the electrosurgical forceps of any of embodiments 1-15, comprising:

forming a strip of a thermally conductive and electrically conductive material into the pair of tines;

providing a groove in a surface of at least one tine of the pair of tines;

securing the heat pipe within the groove; and

mounting the pair of tines and the at least one terminal to the insulated cap portion.

17. The method of embodiment 16, further comprising placing a thermal interface material within the groove for thermal communication between at least portions of the heat pipe and the body of the at least one tine.
18. The method of any of embodiments 16-17, further comprising forming the tines with a curvature, and bending the heat pipe to conform to the curvature of the at least one tine.
19. The method of any of embodiments 16-18, further comprising securing a second heat pipe to a second tine of the pair of tines.
20. The method of any of embodiments 16-19, further comprising providing a layer of an insulating material over each tine of the pair of tines.

As used herein, “consisting essentially of” allows the inclusion of materials or steps that do not materially affect the basic and novel characteristics of the claim. Any recitation herein of the term “comprising,” particularly in a description of components of a composition or in a description of elements of a device, can be exchanged with “consisting essentially of” or “consisting of.”

It will be appreciated that the various features of the embodiments described herein can be combined in a variety of ways. For example, a feature described in conjunction with one embodiment may be included in another embodiment even if not explicitly described in conjunction with that embodiment.

To the extent that the appended claims have been drafted without multiple dependencies, this has been done only to accommodate formal requirements in jurisdictions which do not allow such multiple dependencies. It should be noted that all possible combinations of features which would be implied by rendering the claims multiply dependent are explicitly envisaged and should be considered part of the invention.

The present invention has been described in conjunction with certain preferred embodiments. It is to be understood that the invention is not limited to the exact details of construction, operation, exact materials or embodiments shown and described, and that various modifications, substitutions of equivalents, alterations to the compositions, and other changes to the embodiments disclosed herein will be apparent to one of skill in the art.

What is claimed is:

1. An electrosurgical forceps comprising:
an insulated cap portion;
at least one electrical terminal extending from and fixed to the cap portion;
a pair of tines, each tine comprising a generally elongated body, a tip located at a distal end of the tine, and a proximal end of the tine fixed within the cap portion, at least one tine of the pair of tines electrically connected to the at least one electrical terminal within the cap portion, the body of the at least one tine comprising an electrically conductive and thermally conductive material, and having a groove formed within a surface of the body extending from the tip to a location at a midportion of the body; and
a heat pipe secured within the groove of the at least one tine, the heat pipe comprising a sealed casing and a working fluid within the casing for fluid flow between a condenser section and an evaporator section, the evaporator section disposed in thermal communication with the tip, and the condenser section disposed in thermal communication with a portion of the body of the at least one tine at a location proximally spaced from the tip.

2. The forceps of claim 1, further comprising a thermal interface material within the groove in thermal communication between at least portions of the heat pipe and the body of the at least one tine.

3. The forceps of claim 1, wherein the groove is disposed in an inwardly facing surface of the at least one tine.

4. The forceps of claim 1, wherein the heat pipe is straight.

5. The forceps of claim 1, wherein the heat pipe includes a curved or bent section conforming to a curve or bend in the at least one tine.

6. The forceps of claim 1, wherein the heat pipe has a circular, oval, elliptical, or flattened cross section.

7. The forceps of claim 1, wherein the casing of the heat pipe is copper or a copper alloy.

8. The forceps of claim 1, wherein the working fluid is water.

9. The forceps of claim 1, wherein the heat pipe includes a wicking structure within the casing, the wicking structure comprising a plurality of capillary channels for liquid flow of the working fluid from the condenser section to the evaporator section.

10. The forceps of claim 1, further comprising an insulating coating over the body of the tine extending from the cap portion to a location adjacent to the tip, the insulating coating extending over the heat pipe.

11. The forceps of claim 1, further comprising a second heat pipe secured within a groove in a second tine of the pair of tines.

12. The forceps of claim 1, wherein the electrically conductive and thermally conductive material of the body of the at least one tine is a stainless steel or aluminum, copper, nickel, titanium, or an alloy thereof.

13. The forceps of claim 1, wherein the tip or the body of the at least one tine or both the tip and the body comprise a composite material, wherein the composite material is a dispersion strengthened silver or copper composite material.

14. The forceps of claim 1, wherein the tip or the body of the at least one tine or both the tip and the body comprise a composite material having aligned elongated nickel particles interspersed in a matrix of silver particles.

15. The forceps of claim 1, wherein the forceps is a bipolar forceps.

16. A method of manufacturing the electrosurgical forceps of claim 1, comprising:
forming a strip of a thermally conductive and electrically conductive material into the pair of tines;
providing a groove in a surface of at least one tine of the pair of tines;
securing the heat pipe within the groove; and
mounting the pair of tines and the at least one terminal to the insulated cap portion.

17. The method of claim 16, further comprising placing a thermal interface material within the groove for thermal communication between at least portions of the heat pipe and the body of the at least one tine.

18. The method of claim 16, further comprising forming the tines with a curvature, and bending the heat pipe to conform to the curvature of the at least one tine.

19. The method of claim 16, further comprising securing a second heat pipe to a second tine of the pair of tines.

20. The method of claim 16, further comprising providing a layer of an insulating material over each tine of the pair of tines.
